(19) United States
(12) Patent Application Publication
(10) Pub. No.: US 2024/0352220 A1
(43) Pub. Date: Oct. 24, 2024

(54) MEDICAL RUBBER COMPOSITION AND MEDICAL RUBBER PRODUCT

(71) Applicant: Sumitomo Rubber Industries, Ltd., Kobe-shi (JP)

(72) Inventors: Shumpei MORITA, Kobe-shi (JP); Yuichiro MATSUTANI, Kobe-shi (JP)

(73) Assignee: Sumitomo Rubber Industries, Ltd., Kobe-shi (JP)

(21) Appl. No.: 18/633,901

(22) Filed: Apr. 12, 2024

(30) Foreign Application Priority Data
Apr. 21, 2023 (JP) 2023-069790

(51) Int. Cl. C08K 3/22; A61B 5/15; A61J 1/14; A61M 5/315; C08K 3/04; C08K 3/30; C08K 3/34

(52) U.S. Cl. CPC C08K 3/22; A61B 5/150351; A61J 1/1412; A61J 1/1468; A61M 5/31501; C08K 3/04; C08K 3/30; C08K 3/34; A61M 2005/31508; C08K 2003/222; C08K 2003/2237; C08K 2201/003; C08K 2201/006; C08K 2201/014

(57) ABSTRACT

A medical rubber composition can include: a base polymer containing halogenated isobutylene-isoprene-rubber; and an acid acceptor, and a content of the acid acceptor can be 1 part by mass to 3 parts by mass with respect to 100 parts by mass of the base polymer, and a BET specific surface area of the acid acceptor can be 150 m²/g to 200 m²/g.

### CROSS-REFERENCE TO RELATED APPLICATION(S)

The present application claims priority to Japanese patent application JP 2023-069790, filed on Apr. 21, 2023, the entire content of which is incorporated herein by reference in its entirety.

### BACKGROUND

### Field

The present disclosure relates to a medical rubber composition and/or a medical rubber product, for instance, relating to a technique for improving non-elution properties of a medical rubber product.

### Background Art

Medical rubber products to be applied to pharmaceutical containers may be required to have high quality properties and high physical properties. For example, the quality and properties that may be required for a medical rubber plug for sealing or plugging an opening of a vial in which a formulation such as an antibiotic is stored should comply with Test for Rubber Closure for Aqueous Infusions in the 17th edition of the Japanese Pharmacopoeia in order to use the medical rubber plug. Furthermore, it may be essential that the medical rubber plug for, for example, sealing an opening of a vial, has many properties such as gas permeation resistance, non-elution properties, high cleanability, chemical resistance, needle penetration resistance, self-sealability, and high slidability.

For medical rubber products such as medical rubber plugs, halogenated isobutylene-isoprene-rubber can be used because of its excellent gas (air, water vapor) permeation resistance. If heat is applied during kneading and molding of the halogenated isobutylene-isoprene-rubber, halogen gas can be generated, so that equipment may be corroded. An acid acceptor has been known to be blended in the halogenated isobutylene-isoprene-rubber in order to accept an acid in the halogen gas.

For example, Japanese Laid-Open Patent Publication No. 2021-80370 describes a medical rubber composition that contains (a) a base polymer containing halogenated isobutylene-isoprene-rubber and (b) a liquid polymer, and further contains hydrotalcite or magnesium oxide as an acid acceptor.

Japanese Laid-Open Patent Publication No. 2021-53988 describes a slidable rubber material precursor carrying a silicone-rubber-overcoating coating layer, and the slidable rubber material precursor carrying the silicone-rubber-overcoating coating layer includes: a rubber product that contains at least one rubber component selected from isobutylene-isoprene-rubber, halogenated isobutylene-isoprene-rubber, and chloroprene rubber, a filler, a vulcanizing agent, and a vulcanization aid, and further contains an acid acceptor selected from magnesium oxide, zinc oxide, and natural or synthetic hydrotalcite; and an interfacial modification layer that covers the rubber product, and is obtained by containing an amino-modified silicone compound and/or reacting with surface molecules of the rubber product.

If a medical rubber product obtained by molding a medical rubber composition in which halogenated isobutylene-isoprene-rubber is used is stored in a state of being in contact with a pharmaceutical preparation (for example, saline), a pH of the pharmaceutical preparation (for example, saline) may become higher according to the storage period being longer.

### SUMMARY

An aspect of the present disclosure involves a medical rubber composition that can include: a base polymer containing halogenated isobutylene-isoprene-rubber; and an acid acceptor, and an amount of the acid acceptor to be blended is 1 part by mass to 3 parts by mass with respect to 100 parts by mass of the base polymer, and a BET specific surface area of the acid acceptor is 150 m²/g to 200 m²/g.

### BRIEF DESCRIPTION OF THE DRAWINGS

FIG. 1 illustrates a medical rubber product (stopper) according to one or more embodiments of the present disclosure;

FIG. 2 illustrates a medical rubber product (rubber plug) according to one or more embodiments of the present disclosure;

FIG. 3 illustrates a medical rubber product (vial rubber plug) according to one or more embodiments of the present disclosure; and

FIG. 4 illustrates a medical rubber product (rubber plug for vacuum blood collecting tube) according to one or more embodiments of the present disclosure.

### DETAILED DESCRIPTION

One or more embodiments of the present disclosure have been made in view of the aforementioned circumstances in the Background section, and an object of one or more embodiments of the present disclosure, among one or more objects, can be to provide a medical rubber composition that has an acid acceptor blended in halogenated isobutylene-isoprene-rubber, which can allow a pH of a pharmaceutical preparation to be maintained while inhibiting corrosion of equipment due to halogen gas. Additionally or alternatively, an object of one or more embodiments of the present disclosure, among one or more objects, can be to provide a medical rubber product having excellent non-elution properties.

The inventors of the present disclosure investigated the reason why a medical rubber product obtained by molding a conventional medical rubber composition in which an acid acceptor was blended in halogenated isobutylene-isoprene-rubber caused increase of a pH of a pharmaceutical preparation according to the storage period being longer, and have found that the acid acceptor can be eluted into the pharmaceutical preparation and affects the pH. The inventors of the present disclosure have found that, in a case where the BET specific surface area of the acid acceptor is specified as 150 m²/g to 200 m²/g, and an amount of the acid acceptor to be blended is set as 1 part by mass to 3 parts by mass with respect to 100 parts by mass of the base polymer, the obtained medical rubber composition can allow a pH of the pharmaceutical preparation to be maintained while inhibiting corrosion of equipment due to halogen gas.

One or more embodiments of the present disclosure can provide the medical rubber composition that can allow a pH of the pharmaceutical preparation to be maintained while inhibiting corrosion of equipment due to halogen gas. Additionally or alternatively, one or more embodiments the present disclosure can provide the medical rubber product having excellent non-elution properties.

### <Medical Rubber Composition>

A medical rubber composition according to one or more embodiments of the present disclosure can contain a base polymer containing halogenated isobutylene-isoprene-rubber, and an acid acceptor, and an amount of the acid acceptor to be blended is 1 part by mass to 3 parts by mass with respect to 100 parts by mass of the base polymer, and a BET specific surface area of the acid acceptor is 150 m²/g to 200 m²/g.

### (Base Polymer)

Firstly, the base polymer that contains halogenated isobutylene-isoprene-rubber and that can be used in one or more embodiments of the present disclosure will be described. Examples of the halogenated isobutylene-isoprene-rubber can include a chlorinated isobutylene-isoprene-rubber, a brominated isobutylene-isoprene-rubber, and a bromide of a copolymer of isobutylene and p-methylstyrene. As the halogenated isobutylene-isoprene-rubber, the chlorinated isobutylene-isoprene-rubber or the brominated isobutylene-isoprene-rubber may be preferable. The chlorinated isobutylene-isoprene-rubber or the brominated isobutylene-isoprene-rubber can be, for example, obtained by addition of or substitution with chlorine or bromine at an isoprene structure moiety in isobutylene-isoprene-rubber, specifically, at a double bond and/or a carbon atom adjacent to a double bond. The isobutylene-isoprene-rubber can be a copolymer obtained by polymerizing isobutylene and a small amount of isoprene.

A halogen content in the halogenated isobutylene-isoprene-rubber can be preferably 0.5 mass % or more, more preferably 1 mass % or more, and even more preferably 1.5 mass % or more, and preferably 5 mass % or less, more preferably 4 mass % or less, and even more preferably 3 mass % or less.

Specifically, the chlorinated isobutylene-isoprene-rubber can be, for example, at least one of CHLOROBUTYL 1066 [stabilizer: NS, halogen content: 1.26%, Mooney viscosity: 38 ML₁₊₈ (125° C.), specific gravity: 0.92] manufactured by JAPAN BUTYL Co., Ltd., and/or LANXESS X_BUTYL CB 1240 manufactured by LANXESS.

Specifically, the brominated isobutylene-isoprene-rubber can be, for example, at least one of BROMOBUTYL 2255 [stabilizer: NS, halogen content: 2.0%, Mooney viscosity: 46 ML₁₊₈ (125° C.), specific gravity: 0.93] manufactured by JAPAN BUTYL Co., Ltd., and/or LANXESS X_BUTYL BBX2 and X_BUTYL BB 2030 manufactured by LANXESS.

In one or more embodiments of the present disclosure, as the halogenated isobutylene-isoprene-rubber, the chlorinated isobutylene-isoprene-rubber or the brominated isobutylene-isoprene-rubber may be preferably used.

The base polymer may contain a rubber component other than the halogenated isobutylene-isoprene-rubber. Examples of the other rubber component can include butyl-based rubber, isoprene rubber, butadiene rubber, styrene butadiene rubber, natural rubber, chloroprene rubber, nitrile-based rubber such as acrylonitrile butadiene rubber, hydrogenated nitrile rubber, norbornene rubber, ethylene-propylene rubber, ethylene-propylene-diene rubber, acrylic rubber, ethylene-acrylate rubber, fluororubber, chlorosulfonated polyethylene rubber, epichlorohydrin rubber, silicone rubber, urethane rubber, polysulfide rubber, phosphazene rubber, and 1,2-polybutadiene. One of them may be used alone, or two or more of them may be used in combination.

In a case where the other rubber component is used, a content of the halogenated isobutylene-isoprene-rubber in the base polymer can be preferably 90 mass % or more, more preferably 95 mass % or more, and even more preferably 98 mass % or more. It may also be preferable that the base polymer is formed of the halogenated isobutylene-isoprene-rubber.

### (Acid Acceptor)

Next, the acid acceptor used in one or more embodiments of the present i disclosure will be described. The acid acceptor may be blended, for instance, in order to absorb halogen gas (for example, hydrogen chloride gas or hydrogen bromide gas) generated during crosslinking of the halogenated isobutylene-isoprene-rubber. The acid acceptor may not be particularly limited, for instance, as long as the acid acceptor can be blended in the medical rubber composition, and examples of the acid acceptor can include metal oxides, metal hydroxides, and hydrotalcite.

Specific examples of the metal oxide can magnesium oxide (MgO), zinc oxide (ZnO), calcium oxide (CaO), and aluminum oxide (Al₂O₃). Specific examples of the metal hydroxide can include magnesium hydroxide (Mg(OH)₂), zinc hydroxide (Zn(OH)₂), calcium hydroxide (Ca(OH)₂), and aluminum hydroxide (Al(OH)₃). Specific examples of the hydrotalcite can include Mg—Al-based hydrotalcites such as Mg_{4.5}Al₂(OH)₁₃CO₃·3.5H₂O, Mg_{4.5}Al₂(OH)₁₃CO₃, Mg₄Al₂(OH)₁₂CO₃·3.5H₂O, Mg₆Al₂(OH)₁₆CO₃·4H₂O, MgsAl₂(OH)₁₄CO₃·4H₂O, and Mg₃Al₂(OH)₁₀CO₃·1.7H₂O. Either of natural hydrotalcite or synthetic hydrotalcite may be used. One of these acid acceptors may be used alone, or two or more of them may be used in combination.

In one or more embodiments of the present disclosure, the acid acceptor can be preferably the metal oxide and/or metal hydroxide, can be more preferably at least one selected from the group consisting of MgO, ZnO, Ca(OH)₂, and Al(OH)₃, and can be particularly preferably MgO or ZnO.

Specifically, the magnesium oxide used as the acid acceptor can be, for example, a commercially available product such as “STARMAG R” manufactured by Konoshima Chemical Co., Ltd., as but one example.

The BET specific surface area of the acid acceptor used in one or more embodiments of the present disclosure can be preferably 150 m²/g or more, more preferably 160 m²/g or more, and even more preferably 170 m²/g or more, and preferably 200 m²/g or less, more preferably 190 m²/g or less, and even more preferably 180 m²/g or less. In a case where the BET specific surface area of the acid acceptor is within the above-described range, halogen gas generated during processing can be appropriately absorbed by the acid acceptor. The BET specific surface area of the acid acceptor can represent a value measured by a gas phase adsorption method in which nitrogen gas is used as adsorbed gas. For example, the BET specific surface area can be measured according to the measurement method specified in JIS Z 8830:2013 “Determination of the specific surface area of powders (solids) by gas adsorption-BET method.”

The maximum particle diameter of the acid acceptor used in one or more embodiments of the present disclosure can be preferably less than 20 μm, more preferably 18 μm or less, and even more preferably 15 μm or less. In a case where the maximum particle diameter of the acid acceptor is less than 20 μm, defects where the materials themselves become white spot(s) of foreign material can be reduced, and outer appearance of the product can become better. The maximum particle diameter of the acid acceptor can be measured by, for example, a laser diffraction/scattering method (ultrasonication).

The average particle diameter of the acid acceptor used in one or more embodiments of the present disclosure can be preferably 2.0 μm or more and more preferably 3.0 μm or more, and preferably 5.0 μm or less and more preferably 4.0 μm or less. In a case where the average particle diameter of the acid acceptor is within the above-described range, when the acid acceptor is dispersed in the base polymer, aggregation of the acid acceptor may not cause white spot defects, and the acid acceptor can be more uniformly dispersed. The average particle diameter of the acid acceptor can be measured by, for example, a laser diffraction/scattering method (ultrasonication). For this average particle diameter, the volume-based D₅₀ value may be used as the average particle diameter (μm).

An amount of the acid acceptor to be blended in the medical rubber composition of one or more embodiments of the present disclosure can be preferably 1.0 part by mass or more, more preferably 1.2 parts by mass or more, and even more preferably 1.5 parts by mass or more, with respect to 100 parts by mass of the base polymer, and preferably 3.0 parts by mass or less, more preferably 2.5 parts by mass or less, and even more preferably 2.0 parts by mass or less, with respect to 100 parts by mass of the base polymer. In a case where the content of the acid acceptor is 1.0 part by mass or more, the acid accepting effect with respect to halogen gas generated during the rubber kneading/molding processes may be enhanced, and corrosion of equipment used in these processes due to halogen gas can be inhibited. In a case where the content of the acid acceptor is 3.0 parts by mass or less, for instance, the acid acceptor may be inhibited from being eluted from the medical rubber product into a pharmaceutical preparation, and, furthermore, processability (appropriateness of Mooney viscosity) during molding may also become better.

A molar ratio (halogen/acid acceptor) of halogen in the halogenated isobutylene-isoprene-rubber to the acid acceptor can be preferably 1.0 or less, more preferably 0.8 or less, and even more preferably 0.7 or less, and preferably 0.3 or more, more preferably 0.4 or more, and even more preferably 0.5 or more. In a case where the molar ratio (halogen/acid acceptor) is 1.0 or less, for instance, the acid accepting effect with respect to halogen gas generated in the rubber kneading/molding processes can be further enhanced, and corrosion of equipment used in these processes due to halogen gas can be further inhibited. In a case where the molar ratio (halogen/acid acceptor) is 0.3 or more, for instance, the acid acceptor can be further inhibited from being eluted from the medical rubber product into a pharmaceutical preparation, and, furthermore, processability (appropriateness of Mooney viscosity) during molding may also become better. The molar ratio (halogen/acid acceptor) can be calculated according to the following equation.

According to one or more embodiments, molar ratio (halogen/acid acceptor)={(parts by mass of halogenated isobutylene-isoprene-rubber×halogen content in halogenated isobutylene-isoprene-rubber)/halogen molecular weight}/{parts by mass of acid acceptor/acid acceptor molecular weight}

### (Vulcanizing Agent)

Preferably, the medical rubber composition of one or more embodiments of the present disclosure can further contain a vulcanizing agent. The vulcanizing agent can be blended in order to vulcanize the halogenated isobutylene-isoprene-rubber component contained in the base polymer. The vulcanizing agent may not be particularly limited as long as the halogenated isobutylene-isoprene-rubber can be vulcanized by the vulcanizing agent. Examples of the vulcanizing agent can include sulfur, organic peroxides, and triazine derivatives. One of them may be used alone, or two or more of them may be used in combination.

Examples of the sulfur can include powdery sulfur, sulfur fine powder, precipitated sulfur, colloidal sulfur, and sulfur chloride.

Examples of the organic peroxide can include a dialkyl peroxide, a peroxy ester, a peroxy ketal, and a hydroperoxide. Examples of the dialkyl peroxide include di(2-t-butylperoxyisopropyl) benzene, dicumyl peroxide, 2,5-dimethyl-2,5-di(t-butylperoxy) hexane, t-butylcumyl peroxy, di-t-hexylperoxy, di-t-butylperoxy, and 2,5-dimethyl-2,5-di(t-butylperoxy) hexyne-3. Examples of the peroxy ester include t-butylperoxy maleate, t-butylperoxy-3,3,5-trimethyl cyclohexanoate, t-butylperoxy laurate, t-butylperoxyisopropyl monocarbonate, t-hexylperoxy benzoate, 2,5-dimethyl-2,5-di(benzoylperoxy) hexane, t-butylperoxy acetate, and t-butylperoxy benzoate. Examples of the peroxy ketal include 1,1-di(t-hexylperoxy)-3,3,5-trimethylcyclohexane, 1,1-di(t-hexylperoxy)cyclohexane, 1,1-di(t-butylperoxy)-2-methylcyclohexane, 1,1-di(t-butylperoxy)cyclohexane, 2,2-di(t-butylperoxy) butane, n-butyl-4,4-di(t-butylperoxy) valerate, and 2,2-di(4,4-di(t-butylperoxy)cyclohexyl) propane. Examples of the hydroperoxide include p-menthane hydroperoxide and diisopropylbenzene hydroperoxide.

Examples of the triazine derivative can include compounds represented by general formula (1).

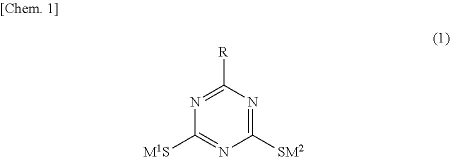

[in the formula, R represents —SH, —OR¹, —SR², —NHR³, or —NR⁴R⁵ (R¹, R², R³, R⁴, and R⁵ each represent an alkyl group, an alkenyl group, an aryl group, an aralkyl group, an alkylaryl group, or a cycloalkyl group. R⁴ and R⁵ may be the same or different from each other.). M¹ and M² can each represent H, Na, Li, K, ½ Mg, ½Ba, ½Ca, an aliphatic primary amine, secondary amine, or tertiary amine, a quaternary ammonium salt, or a phosphonium salt. M¹ and M² may be the same or different from each other.]

In the general formula (1), examples of the alkyl group can include a C1 to C12 alkyl group such as a methyl group, an ethyl group, an n-propyl group, an isopropyl group, an n-butyl group, an isobutyl group, a tert-butyl group, an n-pentyl group, an isopentyl group, a tert-pentyl group, an n-hexyl group, a 1,1-dimethylpropyl group, an octyl group, an isooctyl group, a 2-ethylhexyl group, a decyl group, and a dodecyl group. Examples of the alkenyl group can include a C1 to C12 alkenyl group such as a vinyl group, an allyl group, a 1-propenyl group, an isopropenyl group, a 2-butenyl group, a 1,3-butadienyl group, and a 2-pentenyl group. Examples of the aryl group can include monocyclic or condensed polycyclic aromatic hydrocarbon groups, and can include a C6 to C14 aryl group such as a phenyl group, a naphthyl group, an anthryl group, a phenanthryl group, and an acenaphthylenyl group. Examples of the aralkyl group can include a C7 to C19 aralkyl group such as a benzyl group, a phenethyl group, a diphenylmethyl group, a 1-naphthylmethyl group, a 2-naphthylmethyl group, a 2,2-diphenylethyl group, a 3-phenylpropyl group, a 4-phenylbutyl group, a 5-phenylpentyl group, a 2-biphenylylmethyl group, a 3-biphenylylmethyl group, and a 4-biphenylylmethyl group. Examples of the alkylaryl group can include a C7 to C19 alkylaryl group such as a tolyl group, a xylyl group, and an octylphenyl group. Examples of the cycloalkyl group can include a C3 to C9 cycloalkyl group such as a cyclopropyl group, a cyclobutyl group, a cyclopentyl group, a cyclohexyl group, a cycloheptyl group, a cyclooctyl group, and a cyclononyl group.

Specific examples of the triazine derivative represented by the general formula (1) can include 2,4,6-trimercapto-s-triazine, 2-methylamino-4,6-dimercapto-s-triazine, 2-(n-butylamino)-4,6-dimercapto-s-triazine, 2-octylamino-4,6-dimercapto-s-triazine, 2-propylamino-4,6-dimercapto-s-triazine, 2-diallylamino-4,6-dimercapto-s-triazine, 2-dimethylamino-4,6-dimercapto-s-triazine, 2-dibutylamino-4,6-dimercapto-s-triazine, 2-di(iso-butylamino)-4,6-dimercapto-s-triazine, 2-dipropylamino-4,6-dimercapto-s-triazine, 2-di(2-ethylhexyl)amino-4,6-dimercapto-s-triazine, 2-dioleylamino-4,6-dimercapto-s-triazine, 2-laurylamino-4,6-dimercapto-s-triazine, 2-anilino-4,6-dimercapto-s-triazine, and sodium salts or disodium salts thereof.

In one or more embodiments of the present disclosure, sulfur or the triazine derivative may be preferably used as the vulcanizing agent.

A content of the vulcanizing agent in the medical rubber composition of one or more embodiments of the present disclosure can be preferably 0.1 parts by mass or more, more preferably 0.2 parts by mass or more, and even more preferably 0.3 parts by mass or more, with respect to 100 parts by mass of the base polymer, and preferably 5.0 parts by mass or less, more preferably 3.0 parts by mass or less, and even more preferably 1.0 part by mass or less, with respect to 100 parts by mass of the base polymer. In a case where the content of the vulcanizing agent is within the above-described range, rubber having good rubber properties (hardness, tension, Cset) and/or good processability (little burning) can be obtained.

The medical rubber composition of one or more embodiments of the present disclosure can preferably contain no vulcanization accelerator, for instance, because a vulcanization accelerator may remain in a final rubber product and may be eluted into a pharmaceutical liquid in a syringe or vial. Examples of the vulcanization accelerator can include guanidine-based accelerators (e.g. diphenylguanidine), thiuram-based accelerators (e.g. tetramethylthiuram disulfide, tetramethylthiuram monosulfide), dithiocarbamate-based accelerators (e.g. zinc dimethyldithiocarbamate), thiazole-based accelerators (e.g. 2-mercaptobenzothiazole, dibenzothiazyl disulfide), and sulfenamide-based accelerators (e.g. N-cyclohexyl-2-benzothiazole sulfenamide, N-t-butyl-2-benzothiazole sulfenamide).

### (Filler)

The medical rubber composition of one or more embodiments of the present disclosure may further contain a filler. Examples of the filler can include an inorganic filler such as clay and talc, and resin powder of an olefin resin, styrene-based elastomer, or ultrahigh molecular weight polyethylene (UHMWPE). Among them, as the filler, the inorganic filler may be preferable, and clay or talc may be more preferable. The filler can function to adjust rubber hardness of the medical rubber product, and can also function as an extender to reduce production cost of the medical rubber product.

Examples of the clay can include burned clay and kaolin clay. Specific examples of the clay can include SILLITIN (registered trademark) Z manufactured by Hoffmann Mineral, SATINTONE (registered trademark) W manufactured by Engelhard Corporation, NN Kaolin Clay manufactured by Tsuchiya Kaolin Kogyo, and PoleStar 200R manufactured by Imerys Specialities Japan.

Specific examples of the talc can include High toron A manufactured by Takchara Kagaku Kogyo Co., Ltd., MICRO ACE (registered trademark) K-1 manufactured by NIPPON TALC CO., LTD., and ImerFlex (registered trademark) T20 manufactured by Imerys Specialities Japan.

A content of the filler in the medical rubber composition of one or more embodiments of the present disclosure can be preferably set as appropriate according to, for example, the rubber hardness of a target medical rubber product. The content of the filler in the medical rubber composition of one or more embodiments of the present disclosure can be, for example, preferably 5 parts by mass or more, more preferably 10 parts by mass or more, and even more preferably 20 parts by mass or more, with respect to 100 parts by mass of the base polymer, and preferably 200 parts by mass or less, more preferably 150 parts by mass or less, and even more preferably 100 parts by mass or less, with respect to 100 parts by mass of the base polymer.

### (Other Components)

Furthermore, in the medical rubber composition of one or more embodiments of the present disclosure, in addition to the above-described components, a coloring agent such as titanium oxide and carbon black, a lubricant such as stearic acid and low density polyethylene (LDPE), a processing aid, polyethylene glycol acting as a crosslinking activator, and/or the like may be blended at an appropriate proportion or proportions.

The medical rubber composition of one or more embodiments of the present disclosure can be obtained, for instance, by kneading the base polymer, the acid acceptor, and other blending materials to be added as necessary. For example, the kneading can be performed by using an open roll, a sealed kneader, or the like. The kneaded product can be preferably formed into a ribbon-like shape, a sheet-like shape, a pellet-like shape, or the like, and can be more preferably formed into a sheet-like shape.

A Mooney viscosity (ML₁₊₄ (100° C.)) of the medical rubber composition of one or more embodiments of the present disclosure can be preferably 60 or more and more preferably 62 or more, and is preferably 75 or less and more preferably 70 or less. In a case where the Mooney viscosity (ML₁₊₄ (100° C.)) is within the above-described range, kneadability of the medical rubber composition may become better, and processability can be further enhanced.

### <Medical Rubber Product>

One or more embodiments of present disclosure can include a medical rubber product formed by molding the medical rubber composition of one or more embodiments of the present disclosure. The medical rubber product of one or more embodiments of the present disclosure can be produced, for example, where the medical rubber composition according to one or more embodiments of the present disclosure formed into a ribbon-like shape, a sheet-like shape, a pellet-like shape, or the like can be press-molded, thereby obtaining a medical rubber product having a desired shape. A crosslinking reaction (vulcanization reaction) of the medical rubber composition can progress during the pressing process. The molding temperature can be, for example, preferably 130° C. or higher and more preferably 140° C. or higher, and preferably 200° C. or lower and more preferably 190° C. or lower. The molding time can be preferably two minutes or longer and more preferably three minutes or longer, and preferably 60 minutes or shorter and more preferably 30 minutes or shorter. The molding pressure can be preferably 0.1 MPa or more and more preferably 0.2 MPa or more, and preferably 10 MPa or less and more preferably 8 MPa or less.

Any unnecessary portion of a molded product that may be obtained after the press-molding can be cut and removed, and the molded product can have a predetermined shape. The obtained molded product can be washed, sterilized, dried, and packed, thereby producing a medical rubber product.

Furthermore, a resin film may be integrally laminated on the medical rubber product of one or more embodiments of the present disclosure. Examples of the resin film can include an inactive resin film formed of, for example, polytetrafluoroethylene (PTFE), tetrafluoroethylene-ethylene copolymer (ETFE), a modified product thereof, or ultra-high density polyethylene (UHDPE).

The resin film can be, for example, press-molded in a state of being stacked on the sheet-shaped rubber composition, and may thus be integrated with the medical rubber product formed after the press-molding.

The medical rubber product of one or more embodiments of the present disclosure can be suitably used as a medical rubber product that can particularly come into contact with a pharmaceutical preparation, blood, or the like. Specific examples of the medical rubber product of one or more embodiments of the present disclosure can include medical rubber plugs such as rubber plugs of containers for various pharmaceutical preparations such as a liquid preparation, a powder preparation, and a freeze-dried preparation, and rubber plugs for vacuum blood collecting tubes, and sliding or sealing components such as a syringe stopper.

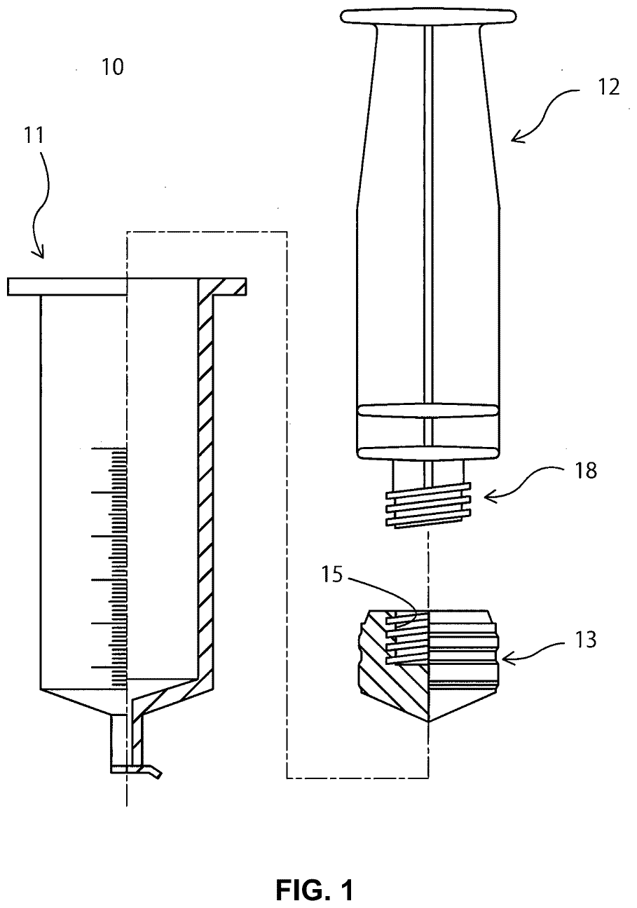

FIG. 1 is an exploded view of a medical syringe, that is, a syringe called a prefillable syringe, for which the medical rubber product of one or more embodiments of the present disclosure may be used. The half of each of a syringe 11 and a stopper 13 is shown as a cross-sectional view in FIG. 1. A prefillable syringe 10 can include the syringe 11 having a cylindrical shape, a plunger 12 that is combined with the syringe 11 and that can reciprocate in the syringe 11, and the stopper 13 attached to the head of the plunger 12. The stopper 13 can be molded from the medical rubber composition of one or more embodiments the present. A laminate film may be stacked on the surface of the stopper 13 in order to improve slidability. And to be clear, components made from the medical rubber composition according to one or more embodiments of the disclosed subject matter may additionally or alternatively be regarded as constituting one or more embodiments of the present disclosure.

The plunger 12 can be, for example, formed of a resin plate having a cross-shaped transverse cross-section, and can have at the head of the plunger 12, a head portion 18 to which the stopper 13 can be attached. The head portion 18 can be formed of resin and integrated with the plunger 12, and can be processed into a male screw shape. The stopper 13 can have a substantially cylindrical shape having a short axis, and the end surface can have, for example, a mountain-like shape forming an obtuse angle and having a protruding axial center portion. The stopper 13 can have a female-screw-shaped fitting recess 15 that can be engraved from the rear end surface in the axial direction. By screwing the head portion 18 of the plunger 12 into the fitting recess 15 of the stopper 13, the stopper 13 can be attached to the head of the plunger 12.

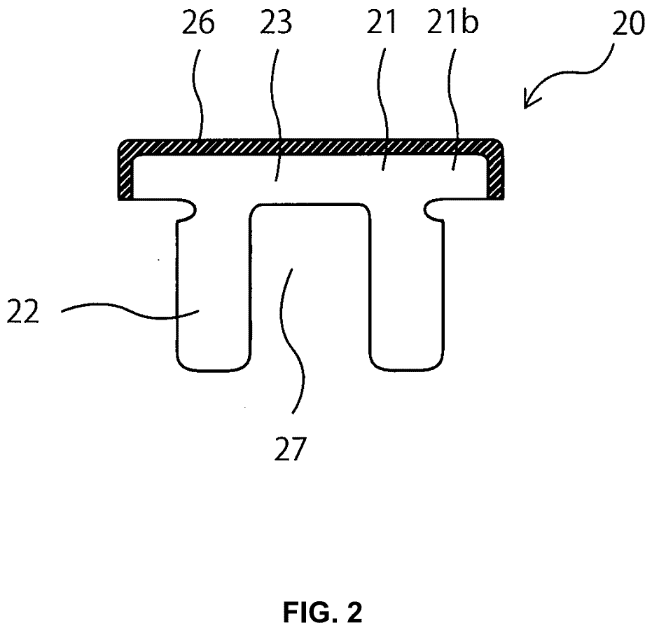

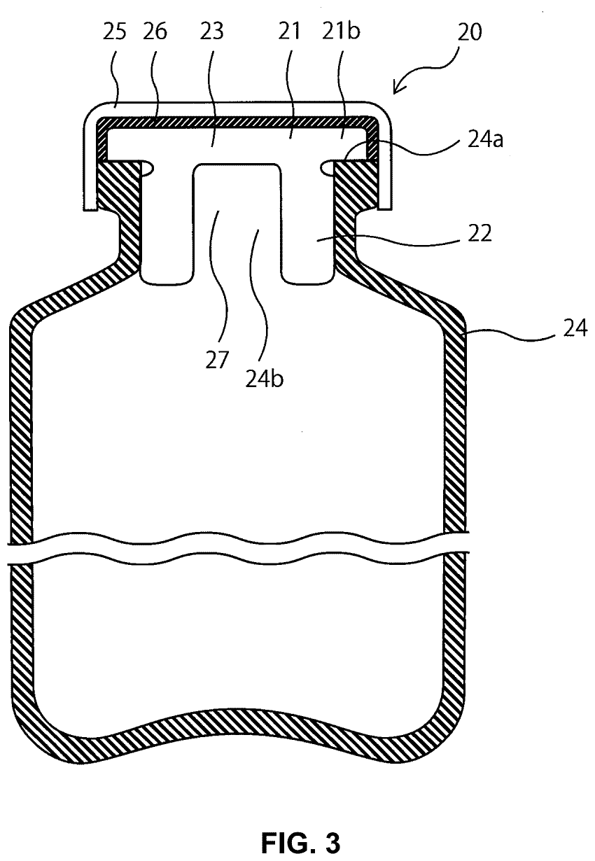

FIG. 2 is a schematic cross-sectional view of one specific example of a rubber plug 20 as the medical rubber product of one or more embodiments of the present disclosure. FIG. 3 illustrates an example of a state where an opening 24b of a container 24 filled with a pharmaceutical preparation is sealed by the rubber plug 20.

The rubber plug 20 can have a top plate 21 and a plug leg 22. The top plate 21 can have a puncture portion 23 that can be punctured with an injector needle of an injector, and a flange portion 21b that can come into contact with an upper edge surface 24a of a container opening of the medical container 24. The plug leg 22 can protrude from the lower surface of the top plate 21, and can be inserted into the medical container opening. The plug leg 22 can have a substantially cylindrical shape, and can have a cut portion 27. A nylon film layer 26 can be disposed on the top surface portion of the top plate 21 of the rubber plug 20. In a case where the nylon film layer 26 is disposed on the top surface portion of the rubber plug 20, mechanical transportability can be ensured while a pharmaceutical preparation can be produced. In a case where the nylon film layer 26 is disposed on the top surface portion of the rubber plug 20, surface lubricity of the top surface portion can be enhanced, and needle puncture fragments can be prevented from being generated during puncturing with an injector needle.

In the state illustrated in FIG. 3, a vial for storing a freeze-dried preparation can be used as the container 24. In a case where the liquid pharmaceutical preparation stored in the container 24 is a pharmaceutical liquid for injection, the puncture portion 23 of the top plate 21 can be punctured with an injector needle of an injector, and the pharmaceutical liquid for injection can be suctioned into the injector without opening the rubber plug 20. The rubber plug 20 may not be opened for instance, in order to prevent a foreign material from being mixed in the pharmaceutical liquid for injection in the container 24.

A metal or resin cap 25 that can cover the opening 24b of the container 24 and the rubber plug 20 can be disposed on the top plate 21. The rubber plug 20 together with the opening 24b can be scaled with the cap 25, for instance, in order to prevent bacteria from being adhered to the puncture portion 23 at a portion that is punctured by an injector needle and mixed into the pharmaceutical liquid for injection through the injector needle. As the type of the cap 25, for example, a flip-off cap, a pull-top cap, or a clean cap can be used. In a case where a large amount of pharmaceutical liquid for injection is used as in hospitals, a clean cap that can be unsealed with one hand and easily operated can be suitably used.

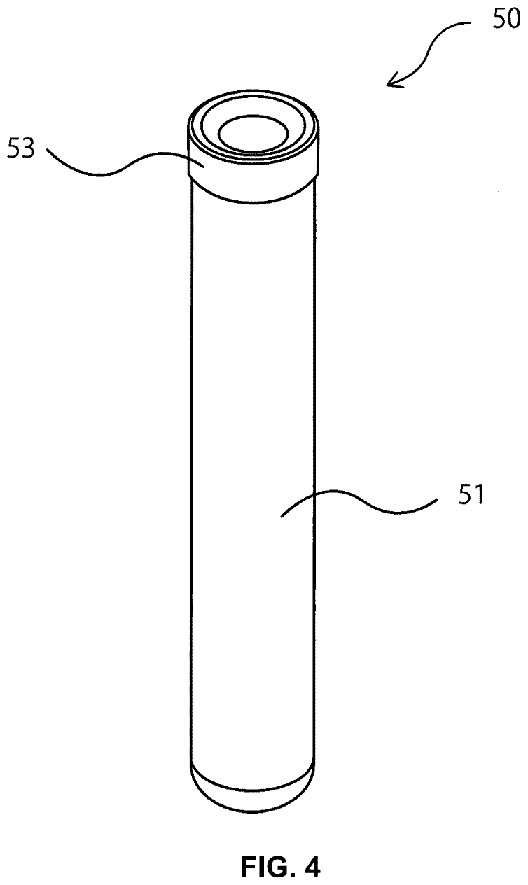

FIG. 4 illustrates one example of a vacuum blood collecting tube. A vacuum blood collecting tube 50 can include a bottomed tube 51, and a rubber plug 53 for seating an opening of the bottomed tube 51. This rubber plug 53 can be formed of the medical rubber composition of one or more embodiments of the present disclosure. The vacuum blood collecting tube 50 can be configured to automatically collect blood, for instance, by reducing pressure in the blood collecting tube.

### Examples

Hereinafter, embodiments of the present disclosure will be described in detail by means of examples, but embodiments of the present disclosure are not limited to the following examples, and any of modifications and implementation modes which do not depart from the gist of the present disclosure can be included in the scope of the present disclosure.

### [Preparation of Medical Rubber Composition]

The materials indicated in Table 1 were kneaded to prepare medical rubber compositions. The materials were kneaded at 80° C. to 140° C. for 5 to 30 minutes by using a sealed-type pressure kneader.

TABLE 1

| Medical rubber composition No. | | | 1 | 2 | 3 | 4 | 5 | 6 | 7 |
|---|---|---|---|---|---|---|---|---|---|
| Blending | Halogenated isobutylene- | | 100 | 100 | 100 | 100 | 100 | 100 | 100 |
| (parts by | isoprene-rubber | | | | | | | | |
| mass) | Acid | Acid acceptor 1 | 12.0 | 9.0 | 6.0 | 5.0 | — | — | 3.0 |
| | acceptor | (BET specific | | | | | | | |
| | | surface area of | | | | | | | |
| | | 178 m²/g) | | | | | | | |
| | | Acid acceptor 2 | — | — | — | — | 1.5 | — | — |
| | | (BET specific | | | | | | | |
| | | surface area of | | | | | | | |
| | | 65 m²/g) | | | | | | | |
| | | Acid acceptor 3 | — | — | — | — | — | 1.5 | — |
| | | (BET specific | | | | | | | |
| | | surface area of | | | | | | | |
| | | 47 m²/g) | | | | | | | |
| | Sulfur | | 0.3 | 0.3 | 0.3 | 0.3 | 0.3 | 0.3 | 0.3 |
| | Talc | | 80 | 80 | 80 | 80 | 80 | 80 | 80 |
| | Titanium oxide | | 3.0 | 3.0 | 3.0 | 3.0 | 3.0 | 3.0 | 3.0 |
| | Carbon black | | 0.3 | 0.3 | 0.3 | 0.3 | 0.3 | 0.3 | 0.3 |
| Evaluation | Mooney viscosity (ML₁₊₄ | | 83 | 78 | 70 | 69 | 62 | 62 | 66 |
| | (100° C.)) | | | | | | | | |
| | Outer appearance | | E | E | E | E | G | P | E |
| | inspection | | | | | | | | |
| | Eluted | Maximum pH | 1.29 | 1.20 | 1.19 | 1.09 | 2.70 | 3.70 | 0.98 |
| | material | difference | | | | | | | |
| | test | between test | | | | | | | |
| | | liquid and blank | | | | | | | |
| | | test liquid | | | | | | | |
| | | Determination | P | P | P | P | P | P | E |

| | | | | | | | |
|---|---|---|---|---|---|---|---|
| Corrosion of equipment | E | E | E | E | E | E | E |

| Medical rubber composition No. | | | 8 | 9 | 10 | 11 | 12 |
|---|---|---|---|---|---|---|---|
| Blending | Halogenated isobutylene- | | 100 | 100 | 100 | 100 | 100.0 |
| (parts by | isoprene-rubber | | | | | | |
| mass) | Acid | Acid acceptor 1 | 2.5 | 2.0 | 1.5 | 1.0 | 0.5 |
| | acceptor | (BET specific | | | | | |
| | | surface area of | | | | | |
| | | 178 m²/g) | | | | | |
| | | Acid acceptor 2 | — | — | — | — | — |
| | | (BET specific | | | | | |
| | | surface area of | | | | | |
| | | 65 m²/g) | | | | | |
| | | Acid acceptor 3 | — | — | — | — | — |
| | | (BET specific | | | | | |
| | | surface area of | | | | | |
| | | 47 m²/g) | | | | | |

| | | | | | | |
|---|---|---|---|---|---|---|
| | Sulfur | 0.3 | 0.3 | 0.3 | 0.3 | 0.3 |
| | Talc | 80 | 80 | 80 | 80 | 80 |
| | Titanium oxide | 3.0 | 3.0 | 3.0 | 3.0 | 3.0 |
| | Carbon black | 0.3 | 0.3 | 0.3 | 0.3 | 0.3 |
| Evaluation | Mooney viscosity (ML₁₊₄ | 64 | 63 | 62 | 61 | 60 |
| | (100° C.)) | | | | | |
| | Outer appearance | E | E | E | E | E |
| | inspection | | | | | |

| | | | | | | |
|---|---|---|---|---|---|---|
| Eluted | Maximum pH | 1.01 | 0.99 | 0.98 | 0.96 | 0.95 |
| material | difference | | | | | |
| test | between test | | | | | |
| | liquid and blank | | | | | |
| | test liquid | | | | | |
| | Determination | E | E | E | E | E |

| | | | | | |
|---|---|---|---|---|---|
| Corrosion of equipment | E | E | G | G | P |

The details of the used blending materials are as follows.

Brominated isobutylene-isoprene-rubber: BROMOBUTYL 2255 manufactured by JAPAN BUTYL Co., Ltd., [bromine content: 2.0%]

Acid acceptor 1: STARMAG (registered trademark) R manufactured by Konoshima Chemical Co., Ltd. (magnesium oxide, BET specific surface area of 178 m²/g, maximum particle diameter of 13 μm, average particle diameter of 3.5 μm)

Acid acceptor 2: MAGSARAT (registered trademark) 150ST manufactured by Kyowa Chemical Industry Co., Ltd. (magnesium oxide, BET specific surface area of 65 m²/g, maximum particle diameter of 20 μm, average particle diameter of 4.8 μm)

Acid acceptor 3: STARMAG (registered trademark) M manufactured by Konoshima Chemical Co., Ltd. (magnesium oxide, BET specific surface area of 47 m²/g, maximum particle diameter of 262 μm, average particle diameter of 3.5 μm)

Sulfur: sulfur containing 5% of oil, manufactured by Tsurumi Chemical Industry Co., Ltd.

Talc: ImerFlex (registered trademark) T20 manufactured by Imerys Specialities

Titanium oxide: TIPAQUE (registered trademark) A-100 manufactured by ISHIHARA SANGYO KAISHA, LTD.

Carbon black: Thermax MT manufactured by Cancarb Limited

### Evaluation Method

### (1) Mooney Viscosity

A Mooney viscosity of the medical rubber composition was measured by a measurement method specified in JIS K6300-1:2013 “Determination of Mooney viscosity and pre-vulcanization characteristics with Mooney viscometer”.

(2) BET Specific Surface Area (m²/g) of Acid Acceptor

The BET specific surface area was measured under the following conditions.
Measurement device: Macsorb HM model (manufactured by Mountech Co., Ltd.)
Pretreatment condition: 160° C., one hour
Method for measuring an amount of adsorbed gas: carrier gas method
Method for analyzing adsorption data: single point method

### (3) Particle Diameter (μm) of Acid Acceptor

In a 100 ml beaker, 50 mL of ethanol was taken, and about 0.2 g of sample powder was put therein, and the obtained product was subjected to ultrasonication (UD-201 manufactured by TOMY SEIKO CO., LTD) for three minutes to prepare a dispersion. The prepared solution was measured by using a laser diffraction method by a particle size distribution analyzer (Microtrac HRA Model 9320-X100 manufactured by NIKKISO CO., LTD.). The volume-based D₅₀ value and the maximum particle diameter were obtained from the obtained particle size distribution.

### (4) Outer Appearance Inspection

The prepared medical rubber composition was press-molded at 15 to 20 atmospheres, at 165° C. to 190° C., for 7 to 20 minutes, to produce a slab (length of 225 mm, width of 60 mm, thickness of 2 mm). The surface of the slab was visually observed, whether or not white spot(s) of foreign material caused due to the acid acceptor was found was determined, and the outer appearance was evaluated according to the following criteria.

Excellent (E): No white spot of foreign material caused due to the acid acceptor was found.

Good (G): Only one white spot of foreign material caused due to the acid acceptor was found.

Poor (P): Two or more white spots of foreign material caused due to the acid acceptor were found.

### (5) Eluted Material Test

The prepared medical rubber composition was press-molded at 15 to 20 atmospheres, at 165° C. to 190° C., for 7 to 20 minutes, to produce a measurement sample (having φ17 mm and a thickness of 2 mm) for an eluted material test. Water-washing in which the produced measurement sample was washed with water or an alkali aqueous solution such as a sodium carbonate aqueous solution at 121° C. for 30 minutes by using an autoclave, and was thereafter immersed in water, was performed three times. In a glass bottle, 200 g of saline was put, and a test liquid in which the washed sample was immersed was stored in a 40° C. oven for predetermined time periods (0 h, 6 h, 24 h, 48 h, 72 h, 96 h, 168 h, 336 h, 504 h). A glass bottle merely containing 200 g of saline as a blank test liquid was also stored in a 40° C. oven for the predetermined time periods. After elapse of the predetermined time periods, the test liquid and the blank test liquid were taken out from the ovens, and cooled to room temperature, a pH of each liquid was thereafter measured by a pH measurement machine (LAQUA D-210P type manufactured by HORIBA, Ltd.), and a difference in pH between the test liquid and the blank test liquid was calculated. The non-elution properties of the acid acceptor were evaluated according to the following criteria by using the maximum value (peak value) of the pH difference between the test liquid and the blank test liquid in the predetermined time periods. The less the pH difference was, the smaller the elution of the acid acceptor was.

Excellent (E): The maximum pH difference between the test liquid and the blank test liquid was less than 1.05.

Poor (P): The maximum pH difference between the test liquid and the blank test liquid was 1.05 or more.

### (6) Corrosion of Equipment

The medical rubber composition was kneaded at 80° C. to 140° C. for 5 to 30 minutes. Thereafter, the metal surface of the kneader was visually checked, and corrosion of the equipment was evaluated according to the following criteria.

Excellent (E): Metal was not corroded, and the luster of the surface was maintained.

Good (G): Metal was slightly corroded, and the luster of the surface was reduced.

Poor (P): Metal was corroded, the luster of the surface was lost, and dirt was adhered and the color changed to brown if the equipment was continuously used.

Table 1 indicates the evaluation result of each item.

As is apparent from the results in Table 1, the medical rubber composition of one or more embodiments of the present disclosure which contained the base polymer containing the halogenated isobutylene-isoprene-rubber, and the acid acceptor, and in which the content of the acid acceptor was 1 part by mass to 3 parts by mass with respect to 100 parts by mass of the base polymer, and the BET specific surface area of the acid acceptor was 150 m²/g to 200 m²/g, was able to inhibit the acid acceptor from being eluted into a pharmaceutical preparation (was able to maintain a pH of the pharmaceutical preparation) while inhibiting corrosion of equipment due to halogen gas.

The present disclosure can be suitably applied to a medical rubber composition and a medical rubber product.

A preferable aspect (1) of one or more embodiments of the present disclosure can be directed to a medical rubber composition including: a base polymer containing halogenated isobutylene-isoprene-rubber; and an acid acceptor, in which an amount of the acid acceptor to be blended is 1 part by mass to 3 parts by mass with respect to 100 parts by mass of the base polymer, and a BET specific surface area of the acid acceptor is 150 m²/g to 200 m²/g.

A preferable aspect (2) of one or more embodiments of the present disclosure can be the medical rubber composition according to the aspect (1) in which the halogenated isobutylene-isoprene-rubber is chlorinated isobutylene-isoprene-rubber or brominated isobutylene-isoprene-rubber.

A preferable aspect (3) of one or more embodiments of the present disclosure can be the medical rubber composition according to the aspect (1) or (2) in which the acid acceptor contains magnesium oxide or zinc oxide.

A preferable aspect (4) of one or more embodiments of the present disclosure can be the medical rubber composition according to any one of the aspects (1) to (3) in which a maximum particle diameter of the acid acceptor is less than 20 μm.

In a preferable aspect (5) of one or more embodiments of the present disclosure, the medical rubber composition according to any one of the aspects (1) to (4) can further include a vulcanizing agent.

In a preferable aspect (6) of one or more embodiments of the present disclosure, the medical rubber composition according to any one of the aspects (1) to (5) can further include a filler.

A preferable aspect (7) of one or more embodiments of the present disclosure can be the medical rubber composition according to any one of the aspects (1) to (6) in which a Mooney viscosity (ML₁₊₄ (100° C.)) is 60 to 75.

A preferable aspect (8) of one or more embodiments of the present disclosure can be directed to a medical rubber product obtained by molding the medical rubber composition according to any one of the aspects (1) to (7).

In a preferable aspect (9) of one or more embodiments of the present disclosure, the medical rubber product according to the aspect (8) can be a stopper for a syringe.

What is claimed is:

1. A medical rubber composition comprising:
a base polymer containing halogenated isobutylene-isoprene-rubber; and
an acid acceptor, wherein
an amount of the acid acceptor to be blended is 1 part by mass to 3 parts by mass with respect to 100 parts by mass of the base polymer, and a BET specific surface area of the acid acceptor is 150 m²/g to 200 m²/g.

2. The medical rubber composition according to claim 1, wherein the halogenated isobutylene-isoprene-rubber is chlorinated isobutylene-isoprene-rubber or brominated isobutylene-isoprene-rubber.

3. The medical rubber composition according to claim 1, wherein the acid acceptor contains magnesium oxide or zinc oxide.

4. The medical rubber composition according to claim 1, wherein a maximum particle diameter of the acid acceptor is less than 20 μm.

5. The medical rubber composition according to claim 1, further comprising a vulcanizing agent.

6. The medical rubber composition according to claim 1, further comprising a filler.

7. The medical rubber composition according to claim 1, wherein a Mooney viscosity (ML₁₊₄ (100° C.)) is 60 to 75.

8. A medical rubber product obtained by molding the medical rubber composition according to claim 1.

9. The medical rubber product according to claim 8, wherein the medical rubber product is a stopper for a syringe.
